(19) United States
(12) Patent Application Publication
(10) Pub. No.: US 2006/0199981 A1
(43) Pub. Date: Sep. 7, 2006

(54) CATALYST

(75) Inventors: Ulf Friedrich Schuchardt, 13025-350 Campinas- SP (BR); Roberto Rinaldi Sobrinho, 13025-350 Campinas-SP (BR)

Correspondence Address: WHITE, REDWAY & BROWN LLP, 1217 KING STREET, ALEXANDRIA, VA, 22314, US

(73) Assignees: FORMIL QUIMICA LTDA., 06612-250 Jandira-SP (BR); UNIVERSIDADE ESTADUAL DE CAMPINAS, 13083-970 Campinas -SP (BR)

(21) Appl. No.: 11/307,326

(22) Filed: Feb. 1, 2006

Related U.S. Application Data
(63) Continuation-in-part of application No. PCT/BR04/00145, filed on Aug. 9, 2004.

(30) Foreign Application Priority Data
Aug. 7, 2003 (BR) PI0305444-6

(51) Int. Cl. C07C 27/04; C07C 29/14

(52) U.S. Cl. 568/862; 568/864

(57) ABSTRACT

The present invention refers to catalysts having high catalytic activity and selectivity, consisting of mixed oxides of copper, aluminum and magnesium and/or calcium, free from chromium, in established proportions. The invention also relates to the preparation thereof. Another object of the present invention is the use of these catalysts in hydrogenation processes, preferably direct hydrogenation of vegetable oil esters to fatty alcohols, represented by formula: CH₃—(CH₂)ₙ—OH, wherein n varies from 7 to 23.

The present invention is a continuation of International Application No. PCT/BR2004/000145, filed Aug. 9, 2004, which claims priority to Brazilian Application PI0305444-6, filed Aug. 7, 2003, both of which are incorporated by reference.

The present invention relates to catalysts for hydrogenation and the preparation of said catalysts. They comprise mixed oxides of copper, aluminum, magnesium and/or calcium, but are free from chromium, and they have high catalytic activity and selectivity for hydrogenation reactions.

Furthermore, the present invention relates to the use of these catalysts in hydrogenation, preferably in direct hydrogenation of vegetable oil esters to fatty alcohols, represented by the formula: CH₃—(CH₂)ₙ—OH, wherein n varies from 7 to 23.

Thus, it is an object of the present invention to produce fatty alcohols by reducing fatty acids esters, obtained from transesterification of vegetable oils, using hydrogenation catalysts that achieve a higher selectivity and a higher yield then known catalysts.

In GB-A-1 600 517 of Chevron Research, it is disclosed that fatty acid esters, obtained by a transesterification of vegetable oils, such as babassu, coconut, soybean, etc., with an alcohol, such as methanol and ethanol, may be hydrogenated by non-catalytic processes for hydrogenating fatty esters, such as reduction with lithium hydrate and metallic aluminum or sodium. However, the process is highly dangerous due to the high activity of the reduction agents, a severe security control being required, both in the hydrogenating process and in the disposal of residues from the process.

The hydrogenation of fatty acid esters by catalytic processes for obtaining fatty alcohols requires suitable conditions so that the hydrogenation reaction can occur with higher conversion rates and higher selectivity.

For many years, several technologies have been designed aiming at operational improvements and improving the catalytic activity of hydrogenation catalyst performance.

Thus, U.S. Pat. No. 1,605,093 describes the use of a copper oxide based catalyst for hydrogenating esters to alcohols, and U.S. Pat. No. 2,091,800 describes catalysts of copper/barium and chromite.

Catalysts consisting of copper and chromium oxide are employed today in chemical industry for direct hydrogenation of fatty acid esters to fatty alcohols.

Several changes in the preparation and in the composition of copper and chromium oxide based catalysts have been made, targeting an improvement in the catalytic activity, the decrease of pressure and temperature of the hydrogenation reaction, and also targeting a higher mechanical and chemical stability of the catalyst.

Accordingly, U.S. Pat. No. 2,782,243, owned by Union Carbide, discloses the use of copper oxide containing 1 to 5 parts of chromium to 100 parts of copper, in the hydrogenation of methyl acetate to alcohol; U.S. Pat. No. 3,173,959 of Dehydag Deutsche describes the production of alcohol by reduction of fatty acids esters in vapor phase, at temperatures ranging from 200 to 300° C. and pressures ranging from 300 to 500 atm, using a mixed catalyst of copper/chromite or copper/zinc/chromite. Moreover, U.S. Pat. No. 4,954,664, owned by Henkel AG, describes the hydrogenation reaction of fats with hydrogen, carried out on catalysts containing from 30 to 40% by weight of copper, 23 to 30% by weight of chromium, 1 to 10% by weight of manganese, 1 to 10% by weight of silicon and 1 to 7% by weight of barium, the oxide/hydroxide catalyst precursor precipitates being converted into oxides by calcination.

It has been found that there is a wide variety of copper and chromium oxide based catalysts available on the market, which are active and selective, thus making possible a great improvement in hydrogenation processes.

In these hydrogenations, pressures of 250 to 300 bar and temperatures of 200 to 400° C. are employed.

However, the disposal of these catalysts is problematic, since chromium is highly toxic. Furthermore, the catalyst amount used is large; the processes make use of about 10% (wt/wt) of catalyst for vegetable oil esters.

According to the present invention, this is overcome, thanks to the catalyst features, the catalyst amount used in relation to the vegetable oil esters being less than that used in the aforementioned technologies.

The new environmental requirements, as established by recent legislation in many countries, each time more and more severe, raise the costs of processes which use copper and chromium, due to the high cost for chromium disposal.

Therefore, the development of new chromium-free catalysts is increasing. For instance, U.S. Pat. No. 4,144,198 discloses the preparation of copper-iron-aluminum-based catalysts to be used in hydrogenation reactions of fatty acid methyl esters, as substitutes for copper oxide/chromite catalysts, and explains the drawbacks of using catalysts related with the hexavalent chromium ions which are discharged as industrial residues from the filtration and washing steps, and which need to be recovered, and with the catalyst particle size, which can be very small, thereby resulting in that the catalyst cannot be effectively separated by filtration from the alcohol obtained after hydrogenation, without the use of diatomaceous earth as filtrating element, raising process costs.

The purpose of chromium in these catalyst types is to stabilize their structures and assure a suitable texture for a high catalytic activity. On the other hand, the presence of copper is important since it is the metal which is responsible for the catalytic activity.

As chromium substitutes, several documents describe the use of titanium, iron, aluminum or zinc, separately or in specific combinations. Generally speaking, other metals can be added in small amounts (less than 10%), in order to optimize the structural characteristics.

Hydrogenation catalysts based on copper and aluminum are known from U.S. Pat. No. 5,053,380, corresponding to BR PI9006513, owned by Union Carbide, which describes the use of these catalysts in processes for hydrogenating organic compounds having carbon-oxygen bonds, to the corresponding alcohols, which catalyst is obtained by co-precipitation of water-soluble copper and aluminum salts, followed by drying and calcination of the precipitate (s) to produce the calcinated catalyst. However, a subsequent activation in a separate reaction is needed, by heating in presence of a reducing gas, with progressive increase of the initial temperature from 50 to 180° C., with a temperature increase rate from 3 to 6° C. per hour. However, in this patent, only a moderate selectivity to alcohol is achieved.

Furthermore, U.S. Pat. No. 5,403,962 of Sud-Chemie describes the use of chromium-free catalysts, based on copper oxide in combination with oxides of zirconium and manganese, presenting high hydrogenating activity, good resistance to acidic components and even to time, without presenting harmful effects to the environment.

It is known that when metals like aluminum or zinc are used (separately or in combination), hydrogenation selectivity decreases due to the formation of saturated hydrocarbons. This reaction may be due to the presence of acidic sites on the catalyst. Therefore, the presence of magnesium in the catalyst formulation prevents the formation of saturated hydrocarbons, thereby increasing selectivity.

The selective hydrogenation of organic compounds containing carbonyl functions, and more precisely, the hydrogenation of esters to alcohols, is also described in U.S. Pat. No. 5,008,235, corresponding to BR PI9006478, owned by Union Carbide, utilizing a catalytic composition obtained by the reduction of a catalyst precursor containing a mixture of copper and aluminum oxides, and also presenting a third component, namely a metal selected from magnesium, zinc, titanium, zirconium, tin, nickel and cobalt, or a mixture thereof, by progressive heating at temperatures ranging from 40 to 150° C., in presence of a reducing gas.

Subsequently, after drying, it is calcinated between 300 and 550° C. According to this patent, the controlled heating of the catalyst aims at providing a high activity and a maximum efficiency in the hydrogenation. It mentions that these catalysts are used for hydrogenating several organic products having a carbonyl function, being mentioned among them the fatty acid esters having aliphatic groups having from 1 to 22 carbon atoms in the alcohol chain. However, there are no examples of such reactions and it is specifically related to dimethyl maleate ester hydrogenation, with low contents of byproducts, such as DES (diethyl succinate).

In U.S. Pat. Nos. 5,658,843 and 5,481,048 is disclosed the reducing activation in two steps, the reduction temperature of the catalytic precursor being a prevailing factor to the catalyst activity and selectivity improvement, the temperature ranging from 20 to 120° C. in a first stage and from 140 to 250° C. in a second stage. These two citations teach that copper oxide based catalysts present low thermal stability and that a fast temperature increase deteriorates catalyst performance, after a certain period of use.

U.S. Pat. No. 5,403,962 and U.S. Pat. No. 5,386,066 teach that a specific relation between the elements is what makes possible to obtain catalysts having modifications in the surface area and a lifetime control, increasing their resistance to acids.

According to the present invention, based on all these teachings and with the purpose of overcoming all the drawbacks of the prior art, there is provided a hydrogenation catalyst having high mechanical and chemical stability and higher selectivity, apt to produce fatty alcohols in high yields.

The present invention makes possible the production of the catalyst, without the need of activation in an independent step, which takes place preferably in situ, that is, in the very reaction mixture, thereby avoiding the state of the art problems.

Another important advantage of the present invention is that the industrial process costs will be lower, the hydrogenation reactions being carried out with an amount of catalyst in the reaction medium ranging from 2.5 to 10% (wt/wt).

In order to achieve these purposes, the present invention sought to provide hydrogenation catalysts, having high mechanical and chemical stability, consisting of mixed oxides of copper, aluminum and magnesium and/or calcium, which are chromium-free and which are highly selective for producing fatty alcohols, in a way that is less aggressive to the environment.

Furthermore, the present invention provides a treatment of the catalyst in such a way that the precursors are calcinated at 400 to 600° C. and present a specific surface area, measured by nitrogen adsorption at-196° C. by the BET [17] method, of about 20 to 200 m²/g.

Thus, the vegetable oil ester hydrogenation catalysts according to the present invention comprise mixed oxides of copper, aluminum and magnesium and/or calcium, having oxygen and/or hydroxyl groups on the surface and oxygen in their structure, enough to provide for an electronically neutral structure. The catalysts have the chemical composition represented by the following formula: CUAl_{b}M_{1.8-b}O_{c} wherein: b is the stoichiometric amount of aluminum in the oxide, wherein b=0 to 1.8; M-is magnesium and/or calcium (Mg being somewhat preferred); c is the stoichiometric amount per formula unity to electrically neutralize the oxide structure. More precisely, c is calculated by (1+1.5·b+(1.8-b))=2.8+0.5·b.

Preferably, b is between 1.4 and 1.0 and even more preferably, between 1.3 and 1.1.

The present invention catalysts may be prepared by any known method, which can assure a highly efficient mixing of the components. The catalysts may be obtained by mixing or wet grinding of copper oxide (s), aluminum oxide (s) and calcium and/or magnesium oxide (s), in suitable proportions, which promotes the texture features of the catalyst, followed by a heat treatment.

Another method is the co-precipitation of suitable precursors employing salt solutions of the metals mentioned in this invention, such as hydroxides, carbonates or basic carbonates, which may be converted to the oxides by calcination.

As pointed out, the catalysts obtained by suitable calcination of the precursors at temperatures ranging from 400° C. to 600° C., present a specific surface of 20 to about 200 m/g, measured by nitrogen adsorption at-196° C. by the BET [17] method.

The activation of the catalysts of the present invention is carried out by reduction with hydrogen. As is well known in the state of the art, the catalyst may be reduced in an atmosphere of hydrogen diluted in an inert gas, such as nitrogen, at a temperature ranging from 100 to 300° C. The precursors, such as hydroxides, carbonates and basic carbonates, may also be directly reduced without being first converted to oxides. The reduction may also be carried out in situ in the hydrogenation reactor, this reduction method being the preferred one.

The catalyst according to the present invention is useful for hydrogenating non-saturated organic substances, in particular compounds having a carbonyl group, such as aldehydes, ketones, carboxylic acids and esters thereof.

The hydrogenation of fatty acid esters, derived from vegetable oils, is a particularly preferred process of the invention. Examples of suitable fatty acids are those which have from 8 to 24 carbon atoms in the carboxylic acid part and from 1 to 4 carbon atoms in the alcoholic part of the ester.

The process of hydrogenating the fatty acid ester according to the present invention consists in bringing into contact the catalyst in its reduced form and the ester at a temperature from 200 to 330° C. and a pressure from 250 to 350 bar. Preferably the process is carried out at 280 to 310° C. at a hydrogen pressure from 280 to 320 bar.

The catalysts of the present invention are characterized in that they present very high catalytic activity, with yields above 90% and a selectivity to alcohol above 99%.

The hydrogenation of fatty acid esters using the catalysts according to the present invention gave fatty alcohols free from hydrocarbons or esters.

It is important to point out that the present invention catalysts have a low content of sodium, less than 1%. The presence of an expressive amount of sodium in the catalyst leads to a decay in the catalytic activity due to the formation of fatty acid salts, which are difficult to hydrogenate.

The interaction between copper and calcium and/or magnesium, in suitable proportions, provides the texture features, resulting in oxides having large specific surface area and a reduction of the atomic mobility of copper over the surface, reducing the catalyst deactivation by sintering, and increases the mechanical and chemical properties of the catalyst, thereby preventing the dissolution of the copper present in the catalyst into the reaction medium.

The object of the present invention will be better understood by means of the examples presented below, which are meant to illustrate a way of carrying out the invention, without restricting or delimiting it to the presented embodiments.

The following examples I to VIII relate to the several formulations of catalysts according to the present invention, using the conditions as set forth according to the invention.

### EXAMPLE I

Preparation of Mixed Oxide of Copper and Aluminum of Chemical Composition CuAl_{1.8}O_{3.7}.

1410 g of Cu (NO₃) 2.3 H₂O and 3942 g of Al (NO₃)₃.9H₂O were dissolved in 10 1 of deionized water, after which the solution was heated to 80° C. This solution of nitrates was added, conjointly, with a 1 mol/l sodium carbonate solution, by means of a pump, to a reactor containing 3 1 of deionized water at 80° C., stirred mechanically at 1000 rpm. The solution addition was carried out during 2 h, in order to maintain the suspension pH between 6.6 to 7.4.

After completion of the solution addition, the precipitate was aged for 2 h at 50° C. under mechanical stirring at 100 rpm. Following this, the precipitate was filtered (thus, contrary to known similar catalysts, the catalayst according to the invention is filterable) and re-suspended using deionized water at 50° C. The precipitate was dried at 80° C. for 12 h and further calcinated at 400° C. under synthetic air flux for 4 h.

### EXAMPLE II

Preparation of Mixed Oxide of Copper, Aluminum and Magnesium of Chemical Composition CuAl_{1.2}Mg_{0.6}O_{3.4}.

1465 g of Cu (NO₃)₂.3H₂O, 2730 g of Al (NO₃)₃.9H₂O and 933 g of Mg (NO₃)₂.6H₂O were dissolved in 10 1 of deionized water, whereafter the solution was heated to 80° C. This nitrate solution was added, conjointly, with a 1 mol/l sodium carbonate solution, by means of a pump, to a reactor containing 3 1 of deionized water at 80° C., stirred mechanically at 1000 rpm. The solution addition was carried out over 2 h, in order to maintain the suspension pH from 6.6 to 7.4. After completion of the solution addition, the precipitate was aged for 2 h at 50° C. under mechanical stirring at 100 rpm. Following, the precipitate was filtered and re-suspended using deionized water at 50° C. The precipitate was dried at 80° C. for 12 h and further calcinated at 400° C. under synthetic air flux for 4 h.

### EXAMPLE III

Preparation of Mixed Oxide of Copper, Aluminum and Magnesium of Chemical Composition CuAl_{0.9}Mg_{0.9}O_{3.25}.

1494 g of Cu (NO₃)₂.3H₂O, 2088 g of Al (NO₃)₃.9H₂O and 1427 g of Mg (NO₃)₂.6H₂O were dissolved in 10 1 of deionized water, and thereafter the solution was heated to 80° C.

This nitrate solution was added, conjointly, with a 1 mol/l sodium carbonate solution, by means of a pump, to a reactor containing 3 1 of deionized water at 80° C., stirred mechanically at 1000 rpm. The solution addition was carried out over 2 h, in order to maintain the suspension pH from 6.6 to 7.4. After completion of the solution addition, the precipitate was aged for 2 h at 50° C. under mechanical stirring at 100 rpm. Then the precipitate was filtered and re-suspended using deionized water at 50° C.

The precipitate was dried at 80° C. for 12 h and further calcinated at 400° C. under synthetic air flux for 4 h.

### EXAMPLE IV

Preparation of Mixed Oxide of Copper, Aluminum and Magnesium of Chemical Composition CuAl_{0.6}Mg_{1.2}O_{3.1}

1525 g of Cu (NO₃)₂-3H₂O and 1420 g of Al (NO₃)₃.9H₂O were dissolved in 10 1 of deionized water, after which the solution was heated to 80° C. This nitrate solution was added, conjointly, with a 1 mol/l sodium carbonate solution, by means of a pump, to a reactor containing 3 1 of deionized water at 80° C., stirred mechanically at 1000 rpm. The solution addition was carried out over 2 h, in order to maintain the suspension pH from 6.6 to 7.4. After completion of the solutions addition, the precipitate was aged for 2 h at 50° C. under mechanical stirring at 100 rpm.

Following this, the precipitate was filtered and re-suspended using deionized water at 50° C. The precipitate was dried at 80° C. for 12 h and further calcinated at 400° C. under synthetic air flux for 4 h.

### EXAMPLE V

Preparation of Mixed Oxide of Copper and Magnesium of Chemical Composition CuMg_{1.8}O_{2.8}.

1589 g of Cu (NO₃)₂.3H₂O and 3035 g of Mg (NO₃)₂.6H₂O were dissolved in 10 1 of deionized water, after which the solution was heated to 80° C. This nitrate solution was added, conjointly, with a 1 mol/l sodium carbonate solution, by means of a pump, to a reactor containing 3 1 of deionized water at 80° C., stirred mechanically at 1000 rpm. The solution addition was carried out over 2 h, in order to maintain the suspension pH from 6.6 to 7.4. After completion of the solutions addition, the precipitate was aged for 2 h at 50° C. under mechanical stirring at 100 rpm.

Following this, the precipitate was filtered and re-suspended using deionized water at 50° C. The precipitate was dried at 80° C. for 12 h and further calcinated at 400° C. under synthetic air flux for 4 h.

### EXAMPLE VI

Preparation of Mixed Oxide of Copper, Aluminum and Calcium of Chemical Composition CuAl_{1.2}Ca_{0.6}O_{3.a}.

1386 g of Cu (NO₃)₂.3H₂O, 2582 g of Al (NO₃)₃.9H₂O and 1122 g of Ca (NO₃)₂.4H₂O were dissolved in 10 1 of deionized water, after which the solution was heated to 80° C. This nitrate solution was added, conjointly, with a 1 mol/l sodium carbonate solution, by means of a pump, to a reactor containing 3 1 of deionized water at 80° C., stirred mechanically at 1000 rpm. The solution addition was carried out over 2 h, in order to maintain the suspension pH from 6.6 to 7.4. After completion of the solution addition, the precipitate was aged for 2 h at 50° C. under mechanical stirring at 100 rpm. Following this, the precipitate was filtered and re-suspended using deionized water at 50° C. The precipitate was dried at 80° C. for 12 h and further calcinated at 400° C. under synthetic air flux for 4 h.

### EXAMPLE VII

Preparation of Mixed Oxide of Copper, Aluminum and Magnesium of Chemical Composition CuAl_{1.2}Mg_{0.6}O_{3.4}.

A solution containing 1465 g of Cu (NO₃)₂.3H₂O, 2730 g of Al (NO₃)₃.9H2O and 933 g of Mg (NO₃)₂.6H₂O in 5 1 of deionized water, and a second solution containing 1607 g of Na₂CO₃ in 5 1 were prepared, and afterwards the solutions were heated to 80° C. The nitrate solution was added to the Na2CO3 solution in a reactor at 80° C., while the suspension pH was kept between 6.6 to 7.4. The precipitate was aged for 2 h at 50° C. under mechanical stirring at 100 rpm.

After this, the precipitate was filtered and re-suspended using deionized water at 50° C. The precipitate was dried at 80° C. for 12 h and further calcinated at 400° C. under synthetic air flux for 4 h.

### EXAMPLE VIII

Preparation of Mixed Oxide of Copper, Aluminum and Magnesium of Chemical Composition CuAl_{1.2}Mg_{0.6}O_{3.4}.

A solution containing 1465 g of Cu (NO₃)₂.6H₂O, 2730 g of Al (NO₃)₃.9 H₂O and 933 g of Mg (NO₃)₂.6H₂O in 5 1 of deionized water, and a second solution containing 1607 g of Na₂CO₃ in 5 1 were prepared, whereafter the solutions were heated to 80° C. The Na₂CO₃ solution was added to the nitrate solution in a reactor at 80° C. under mechanical stirring at 1000 rpm. The mixture was stirred for 2 h at 80° C., while the suspension pH was kept between 6.6 to 7.4. The precipitate was aged for 2 h at 50° C. under mechanical stirring at 100 rpm. Following, the precipitate was filtered and re-suspended using deionized water at 50° C.

The precipitate was dried at 80° C. for 12 h and further calcinated at 400° C. under synthetic air flux for 4 h.

The higher catalytic activity of the mixed oxides catalysts obtained according to the examples I to VIII of the present invention may be confirmed in Table 1, considering the results obtained by the employment of the catalytic mixtures according to present invention in hydrogenation reactions of babassu oil ethylic esters.

It is pointed out that processes according to prior art reduce the surface of the catalyst in a separate process and then transfer the reduced catalyst to the hydrogenation vessel, while according to the present invention, the reduction preferably takes place in the very reaction vessel.

The babassu oil ethylic ester hydrogenation was carried out using the catalysts according to present invention in 2.5% wt/wt at a hydrogen pressure of 300 bar at 300° C. during 1 h of reaction. Yield and selectivity were determined by chromatography.

For the sake of comparison, copper and. chromium based catalysts known from the prior art were used, cf. examples IX and X.

Thus, the following two examples show the state of the art copper and chromium oxide based catalysts, used for hydrogenating fatty acid esters.

### EXAMPLE IX

Fatty Acid Ester Hydrogenation Process.

Hydrogenation of babassu oil ethylic ester was carried out using Engelhard 1150P catalyst (copper and chromium oxide) (2, 5% wt/wt) at a hydrogen pressure of 300 bar at 30. 0° C. during 1 h of reaction. Yield and selectivity were determined by gas chromatography.

### EXAMPLE X

Fatty Acid Esters Hydrogenation Process.

Hydrogenation of babassu oil ethylic ester was carried out using Engelhard 1850P catalyst (copper and chromium oxide) (2, 5% wt/wt) at a hydrogen pressure of 300 bar at 300° C. during 1 h of reaction. Yield and selectivity were measured by gas chromatography.

The performance of the catalysts according to the present invention may be evaluated in Table 1 as compared to catalysts of the state of the art.

TABLE 1

Yield and selectivity to babassu oil ethylic ester hydrogenation reactions using the catalysts of the invention and commercial catalysts (pressure: 300 bar, temperature: 300° C., duration: 1 hour).

CATALYST YIELD SELECTIVITY

Example I (CuAl_{1.8}O_{3.7}) 96 99

Example II (CuAl_{1.2}Mg_{0.6}O_{3.4}) 100 100

Example III (CuAl_{0.9}O_{3.25}) 97 99

Example IV (CuAl_{0.6}Mg_{1.2}O_{3.1}) 97 100

Example V (CuMg_{1.8}O_{2.8}) 97 100

Example VI (CuAl_{1.2}Ca_{0.6}O_{3.4}) 95 99

Example VII (CuAl_{1.2}Mg_{0.6}O_{3.4}) 93 100

Example VIII (CuAl_{1.2}Mg_{0.6}O_{3.4}) 93 99

Example IX (Cu—Cr, Engelhard 1150P) 86 93

Example X (Cu—Cr, Engelhard 1850P) 91 90

1. A catalyst, comprising oxides of copper, aluminum and at least one of magnesium and calcium, said catalyst being free of chromium and having oxygen and/or hydroxyl groups on its surface and oxygen in its structure sufficient to provide for an electronically neutral structure, and having a surface area of from about 20 to about 200 m²/g, measured by nitrogen adsorption at −196° C. by the BET method, the catalyst having the general formula:
CuAl_{b}M_{1.8-b}O_{c}
wherein:
b is the stoichiometric amount of aluminum in the oxide, wherein b=1.0 to 1.4;
M is magnesium and/or calcium;
c is the stoichiometric amount per formula unity to electrically neutralize the oxide structure.

2. Catalyst according to claim 1, wherein b is between 1.1 and 1.3.

3. A process for preparing a catalyst of oxides of copper, aluminum and magnesium and/or calcium, free from chromium, according to claim 1, using salt solutions of the metals, such as hydroxides, carbonates or basic carbonates,
which are converted to the oxides, wherein the suitable calcination of these precursors is carried out at temperatures ranging from 400° C. to 600° C., in order to obtain a surface area of from about 20 to about 200 m²/g, measured by nitrogen adsorption at −196° C. by the BET method.

4. Process according to claim 2, wherein said precursors are directly reduced, in situ, in the hydrogenation reactor.

5. Process according to claim 2, wherein the precursors are obtained by mixing or wet grinding copper oxides, aluminum oxide and calcium and/or magnesium, in suitable proportions, which provides for the texture features of the catalyst and further, if necessary, a heat treatment.

6. Process for hydrogenation of non-saturated organic substances, in particular compounds having carbonyl groups, such as aldehydes, ketones, carboxylic acids and esters thereof using a catalyst according to claim 1 at a temperature from 200° C. to 300° C. and a pressure from 250 bar to 350 bar, wherein said organic substances are contacted, in an amount of catalyst ranging from 2.5 to 10% (wt/wt) in its reduced form, obtained according to any claim 3 to 5, to obtain one or several alcohols.

7. Process according to claim 6, wherein the organic substances are fatty esters having from 8 to 24 carbon atoms in the carboxylic acids part and from 1 to 4 carbon atoms in the ester alcoholic part, which esters are reduced to alcohol (s) of formula CH₃—(CH₂)ₙ—OH, wherein n varies from 7 to 23.

8. Process according to claim 6 wherein the process is carried out at a temperature from 280° C. to 310° C. and a hydrogen pressure from 280 bar to 320 bar.

9. Process according to claim 6, wherein the fatty esters which are hydrogenated, derive from vegetable oils, such as babassu oil, coconut oil and soybean oil.

10. An alcohol or alcohol mixture produced by hydrogenation of fatty esters derived from a vegetable oil, the hydrogenation being performed by a process utilizing a catalyst according to claim 1.
